(19) United States
(12) Patent Application Publication
(10) Pub. No.: US 2016/0175235 A1
(43) Pub. Date: Jun. 23, 2016

(54) SYSTEM AND METHOD FOR EXTENDING, THICKENING AND ENHANCING NATURAL OR ARTIFICIAL HAIR

(71) Applicants: Shani DROR, Tel Aviv (IL); Uzi Lewin, Tel Aviv (IL)

(72) Inventors: Shani DROR, Tel Aviv (IL); Uzi Lewin, Tel Aviv (IL)

(21) Appl. No.: 15/057,137

(22) Filed: Mar. 1, 2016

Related U.S. Application Data
(63) Continuation-in-part of application No. PCT/IL2014/050733, filed on Aug. 14, 2014.

(30) Foreign Application Priority Data
Sep. 2, 2013 (IL) 228243

(51) Int. Cl. A61K 8/88; A61Q 1/10; A61K 8/98; A61K 8/02; A45D 40/26; A41G 5/02

(52) U.S. Cl. CPC A61K 8/88; A45D 40/262; A41G 5/02; A61K 8/985; A61K 8/027; A61Q 1/10; A61K 2800/87; A61K 2800/40

(57) ABSTRACT

The invention relates to a system and method for extending and/or thickening natural or artificial hair, comprising an adhesive material and an additional material suitable to be connected to said natural or artificial hair, which comprises animal hair and/or structurally-modified synthetic fibers having a surface with roughening structures that mimic the surface of animal hair.

### CROSS-REFERENCE TO RELATED APPLICATIONS

The present application is a continuation-in-part application of International Application No. PCT/IL2014/050733, filed Aug. 14, 2014, which claims the benefit of Israeli Patent Application No. 228243, filed on Sep. 2, 2013, the entire contents of each and both these applications being hereby incorporated by reference herein in their entirety as if fully disclosed herein.

### FIELD OF THE INVENTION

The present invention relates to cosmetic applications. More particularly, the invention relates to a system and method for improving and/or altering the appearance of natural or artificial hair, hereinafter “NOAH”, such as eyelashes or eyebrows, by extending and/or thickening them.

### BACKGROUND ART

In modern society it is a common habit, mostly among women (and increasingly among men), to alter the appearance of body hair by coloring, extending, and thickening them with cosmetic products. There are various reasons for coloring the hair, such as styling, concealing gray hair, and repairing damages.

There is also a need for altering the appearance of other NOAH, such as synthetic filaments. The styling of synthetic filaments is common, for instance, in fashion, art, and film industries.

The prior art teaches methods and devices for enhancing the appearance of eyelashes. As described in U.S. Pat. No. 4,135,527 a container of adhesive liquid mascara is provided, along with another container of synthetic fiber filaments. Both substances are applied on the eyelashes, one after the other—first the mascara, and then the filaments. The final result is extended eyelashes, but with no thickening of the hair, and the method described is limited to the use of synthetic fiber filaments, which have the disadvantage of possessing a relatively smooth surface that does not facilitate the required construction and attachment. Therefore, the appearance of the extended eyelashes is only marginally improved, and the added filaments result in a brittle addition that is not long lasting.

The art has so far failed to provide an efficient method and materials, which may substantially improve the appearance of NOAH, and which may provide a long-lasting effect including both substantial lengthening and thickening of the hair.

It is the objects of the present invention to provide a system and method that overcome the drawbacks of the prior art, and provide a long-lasting result.

### SUMMARY OF THE INVENTION

In one aspect, the invention relates to a system for extending and/or thickening NOAH, comprising: an adhesive material and an additional material suitable to be connected to said NOAH, which comprises animal hair and/or structurally-modified synthetic fibers the surface of which has been provided with roughening structures mimicking the surface of animal hair. The NOAH can be, e.g., human hair such as eyelashes or eyebrows, or a synthetic fiber the surface of which has been provided with roughening structures that mimic the surface of animal hair.

In certain embodiments, the additional material comprises animal hair. The animal hair can be any animal hair, e.g., from cat, dog, or rabbit hair. The animal hair can be initially colored.

In certain embodiments, the additional material comprises structurally-modified synthetic fibers having a surface with roughening structures mimicking the surface of animal hair. By “structurally-modified synthetic fibers” it is meant to refer to synthetic fiber the surface of which has been provided with roughening structures that resemble the surface of animal hair, as will be further explained below.

In some particular such embodiments, the structurally-modified synthetic fibers have a thickness in a range of about 10 μm to about 50 μm, e.g., about 10-15, 15-20, 20-25, 25-30, 30-35, 35-40, 40-45, or 45-50 μm. in other particular such embodiments, the surface of the structurally-modified synthetic fibers has roughening structures at all sides, i.e., at all directions of, or all around, the filament, similar to the configuration in animal hair. In further particular such embodiments, the roughening structures have the shape of barbs protruding from the surface of the synthetic fibers.

The adhesive material can be any type of mascara—of any color, water-proof or not water-proof.

The adhesive material and the additional material can be stored and/or used separately, or even be provided mixed together. Accordingly, the adhesive material can be stored in one container and the additional material can be stored in a different container, and the adhesive and additional materials can be applied by different applicator devices, or maybe applied together. Suitable applicator devices can include, for instance, a brush.

In another aspect, the invention relates to the use of animal hair and/or structurally-modified synthetic fibers the surface of which has been provided with roughening structures mimicking the surface of animal hair, for extending and/or thickening NOAH.

In certain embodiments, the animal hair and/or structurally-modified synthetic fibers are used in combination with an adhesive material. In particular such embodiments, the adhesive material used in combination with said animal hair and/or structurally-modified synthetic fibers is mascara.

In a further aspect, the invention relates to a method for extending and/or thickening and/or enhancing NOAH, comprising applying to a NOAH an adhesive material and an additional material comprising animal hair and/or structurally-modified synthetic fibers the surface of which has been provided with roughening structures mimicking the surface of animal hair. In certain embodiments, said adhesive material and said additional material are mixed together prior to applying, and applied together as a mixture. As foresaid, the additional material can be colored before use. In certain embodiments the additional material is mascara.

### BRIEF DESCRIPTION OF THE DRAWINGS

FIG. 1A is a schematic illustration of a nylon fiber.

FIG. 1B is a photograph of a microscopic magnification of a nylon fiber.

FIG. 2A is a schematic illustration of a human hair.

FIG. 2B is a photograph of a microscopic magnification of a human hair.

FIG. 3A is a schematic illustration of a rabbit hair.

FIG. 3B is a photograph of a microscopic magnification of a rabbit hair.

FIG. 4A is a schematic illustration of a cat hair.

FIG. 4B is a photograph of a microscopic magnification of a cat hair.

FIG. 5A is a schematic illustration of a dog hair.

FIG. 5B is a photograph of a microscopic magnification of a dog hair.

FIG. 6A is a schematic illustration of a dog hair of a different origin from the one of FIG. 5A.

FIG. 6B is a photograph of a microscopic magnification of a dog hair of a different origin from the one of FIG. 5B.

FIG. 7A is a photograph of the external structure of thin rabbit hair filaments.

FIG. 7B is a photograph of the external structure of thick rabbit hair filaments.

FIG. 8A is a photograph of thin rabbit hair filaments showing measurements of hair width (of 15.47, 15.46 and 14.60 μm) and of the length of the barbs (9.913, 15.77 and 19.77 μm).

FIG. 8B is a photograph of thick rabbit hair filaments showing measurements of hair width (11.29, 13.02 and 29.12 μm) and of the length of the barbs (12.90, 21.09, and 10.25 μm).

FIG. 9 is a photograph of animal hair as the additional material, which was not colored.

FIG. 10 is a photograph of colored animal hair as the additional material, where the NOAH is eyelashes and the eye is open.

FIG. 11 is a photograph of colored animal hair as the additional material, where the NOAH is eyelashes and the eye is closed.

FIG. 12A is a photograph of a microscopic magnification of part of the invention.

FIG. 12B is another photograph of a microscopic magnification of part of the invention.

### DETAILED DESCRIPTION OF THE INVENTION

The invention relates to a cosmetic product, system and method, for altering NOAH appearance. In the context of this description, a NOAH can be of any type, a hair, such as structurally-modified synthetic fibers, or any other elongated fiber or a material, although in the description to follow reference will be made to eyelashes as the illustrative substrate. The reference to eyelashes, of course, does not mean to limit the invention to a specific use in any way, and the invention can be applied to a broad range of substrates.

In one aspect, the invention relates to a system for extending and/or thickening NOAH, comprising an adhesive material and an additional material suitable to be connected to said NOAH, which comprises animal hair and/or structurally-modified synthetic fibers having a surface with roughening structures mimicking the surface of animal hair.

In certain embodiments, the additional material comprises animal hair. In other embodiments, the additional material comprises structurally-modified synthetic fibers, also referred to herein as synthetic filaments, having a surface with roughening structures mimicking the surface of animal hair. In further embodiments, the additional material comprises a combination of animal hair and structurally-modified synthetic filaments.

The synthetic filaments can be made from suitable materials, e.g., nylon, polydioxanone (PDO), polyglycolic acid (PGA), polyester, polypropylene, silk, and surgical suture materials such as chromic catgut, and plain catgut. Such synthetic filaments are modified by adding roughening structures, e.g., in the shape of barbs, more particularly barbs protruding from the surface of said filaments, so as to mimic the surface of animal hair.

An important feature of animal hair is that the hair filaments have barbs on their surface, which enable them to cling together and produce a longer hair. Certain cog threads or barbed threads are used in cosmetic applications such as facial rejuvenation; however, such synthetic threads have not been used for extending hair, as disclosed herein. Additionally, while the thickness of the cog threads used for cosmetic applications can be as low as a tenth of a millimeter (see www.sinorgmed.com/22.html or www.sinorgmed.com→products→Face Lifting Thread), the synthetic filaments of the invention are generally less than 50 μm thick. Another important difference is in that the barbs of cog threads used for cosmetic applications are generally not protruding and only become protruding after being pulled, whereas the barb-shaped roughening structures of the synthetic filaments used according to the invention are already protruding from the surface of the filament prior to use (mimicking the surface of animal hair), enabling the filaments to combine prior to being used and again during the use, thereby generating a longer addition to the NOAH.

Structurally-modified fibers can be achieved by methods known in the art for obtaining surface modifications of synthetic fibers. Several examples of well-known methods for surface modification are detailed in the document “Surface Modification of PEEK™ Polymer” (available on-line at http://cgtec.eu/wp-content/uploads/Surface-modification-of-PEEK-e.pdf, which describes different methods, such as surface roughening, surface etching, plasma/corona/laser treatments, and UV radiation for achieving surface modification.

Another example of a method for surface modification is described in the document “Cold Gas Plasma Surface Modification Adhesion Bonding.pdf” (available on-line at http://www.adhesionbonding.com/2012/04/30/cold-gas-plasma-surface-modification-optimize-plastics-bonding-adhesion/), which explains the process of surface modification by cold gas plasma.

There are also many references in the art to surface patterning, e.g., in the article “A Survey of Surface Modification Techniques” (an abstract of a review is available on-line at http://www.mdpi.com/2073-4360/6/9/2309).

The structurally-modified fibers of the invention can be achieved by any method known in the art, and the above references are but examples of several techniques available in the art to modify the surface of synthetic materials and do not mean to limit the invention to any specific method for obtaining structurally-modified fibers.

The extension and thickening of NOAH is achieved by applying filament-based material as described above, referred to herein as an additional material on the substrate—NOAH. The system of the present invention comprises an additional material that can be applied in combination with an adhesive material, or which can be adhesive by itself. When the adhesive material and the additional material are separate from one another, first the adhesive material is applied to the NOAH, and then the additional material is applied, to create a layer of added material. The adhesive material can be used again for a second layer, which may partially or essentially fully overlap with the first layer, after the additional material is applied. When the additional material and the adhesive material are the same material or if they are inseparable, they are applied on the filaments in a one-stage application.

Accordingly, in another aspect, the invention relates to the use of animal hair and/or structurally-modified synthetic fibers having a surface with roughening structures mimicking the surface of animal hair, for extending and/or thickening NOAH.

In a further aspect, the invention provides a method for extending and/or thickening and/or enhancing NOAH, comprising applying to a NOAH an adhesive material and an additional material comprising animal hair and/or structurally-modified synthetic fibers having a surface with roughening structures mimicking the surface of animal hair.

In certain embodiments, the adhesive material and the additional material are mixed together prior to application to NOAH; and in other embodiments, the adhesive material is applied to NOAH first, and the additional material is applied afterwards.

In any case, the application of the materials can be performed by any method and instruments, which are suitable to spread the materials on the NOAH. A suitable instrument can be for example, a brush. One example of instruments suitable to apply the additional material on the NOAH is a set of two containers, wherein each of the containers is provided with a brush. The first container comprises mascara that is applied by the first brush, as the adhesive material, and the other container is provided with a second brush for applying the additional material. Such set of containers and brushes contributes to the separation between the mascara and the additional material and prevents a mixture which in some cases may be undesirable, or which may be preferred by some users for convenience of application.

The durability of the final structure is determined by the strength of the connection between the NOAH and the additional material. The durability is very much depended on the friction between the surface of the additional material, and the NOAH, resulting from the geometrical shape of both the additional material and the NOAH—when the friction increases, so is the durability of the final structure. Since the invention relates to the altering of the appearance of a given desired NOAH, the friction is a function of the chosen additional material, and the adhesive material.

In the case of separate additional material and adhesive material, the adhesive material can be any kind of mascara, or any other adhesive material suitable to be applied to the NOAH. As will be apparent to the skilled person, many types of mascara are commercially available and the invention is not limited to any specific type of mascara or of any other adhesive material.

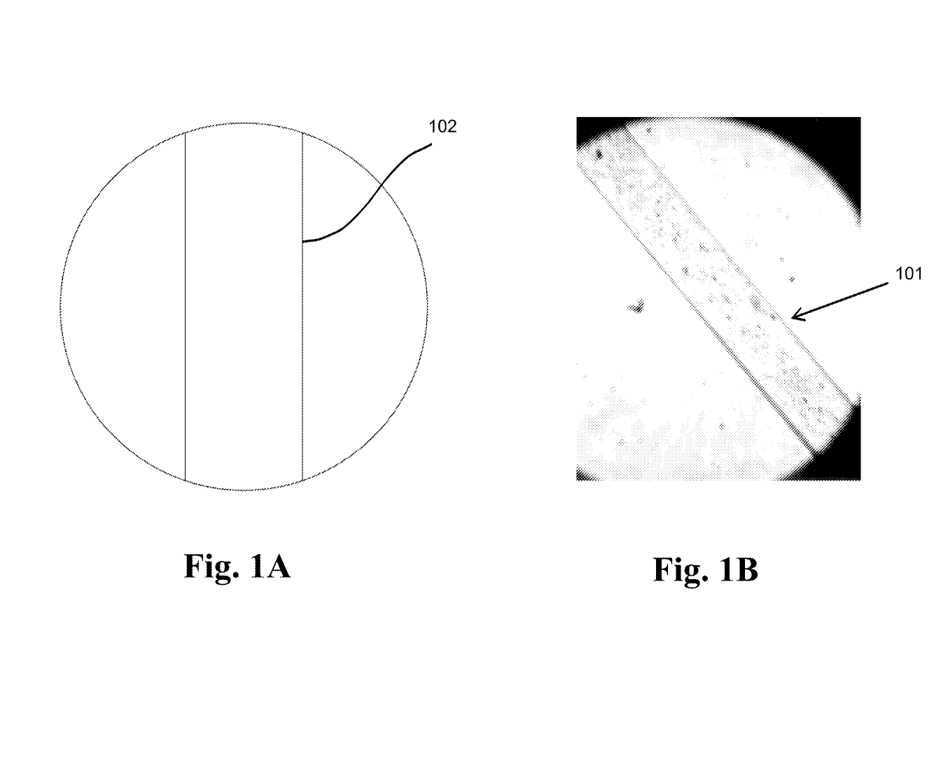

In the figures, a few examples of additional materials are shown. FIG. 1B is a photograph of a microscopic magnification of a nylon fiber 101. FIG. 1A is a schematic drawing of the photograph of FIG. 1B. As shown, the surface 102 of a nylon fiber is essentially smooth, and therefore is geometrically less suitable than other materials to create a durable connection. According to the prior art, synthetic fibers are used to extend eyelashes. As seen in the figures, the geometric shape of the surface of a non-modified synthetic fiber, such as a nylon fiber, is substantially smooth, which makes it unsuitable for the purpose of this invention.

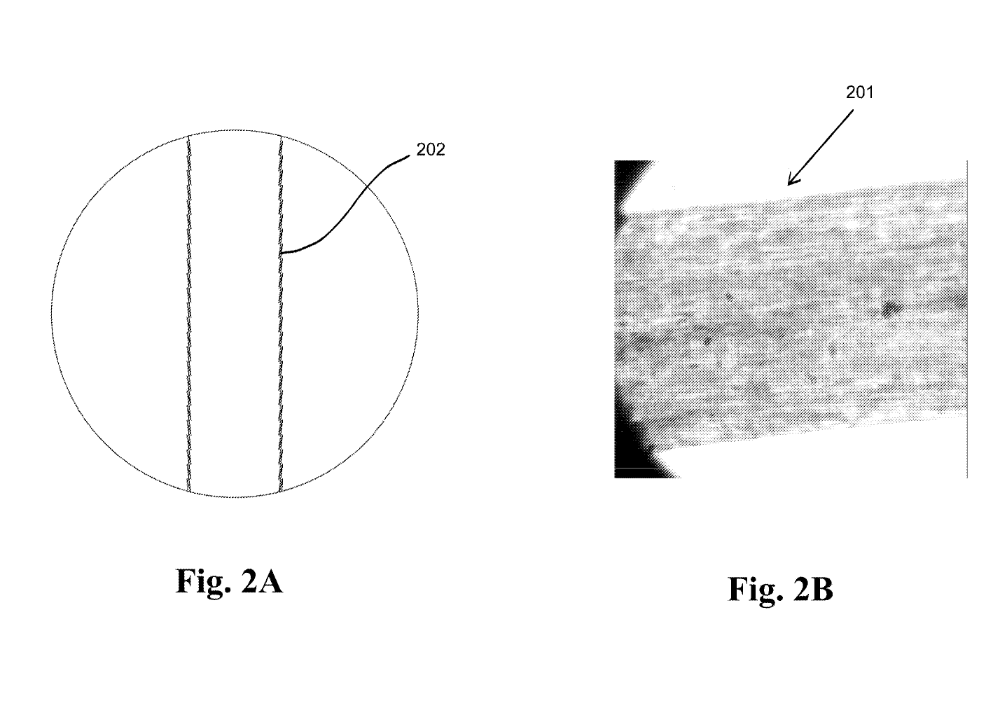

FIG. 2B is a photograph of a microscopic magnification of a human hair 201. FIG. 2A is a schematic drawing of the photograph of FIG. 2B. As shown, the surface 202 of a human hair 201 is less smooth than the surface 102 of the nylon fiber 101.

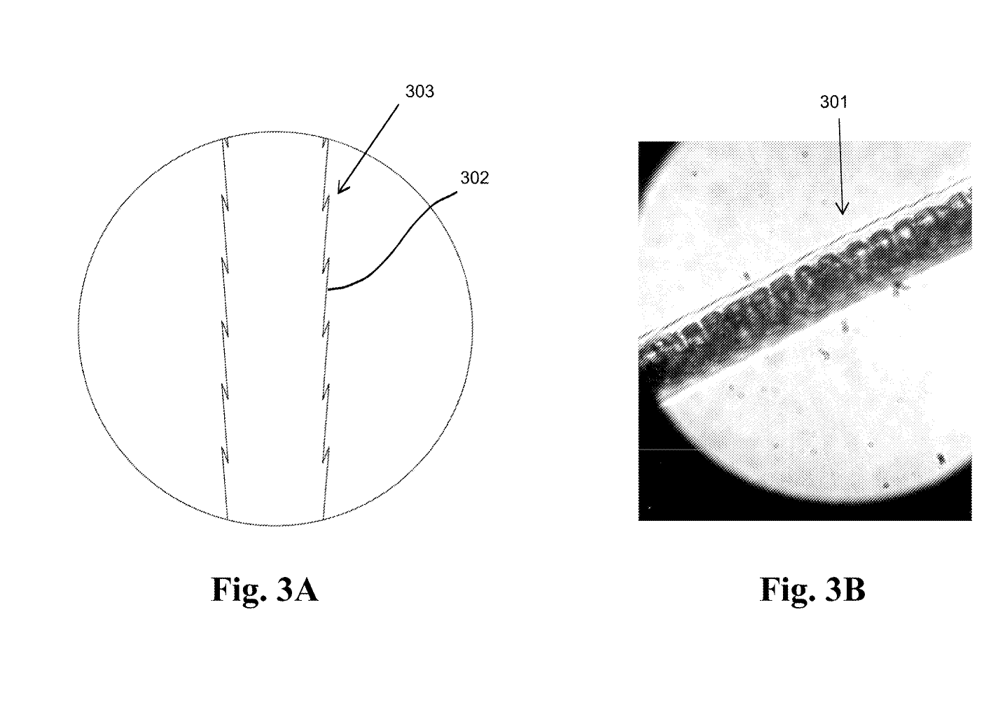

According to the invention, animal hair or structurally-modified synthetic fibers (as herein defined) have a suitable surface, which substantially improves the final result, relative to the prior art. FIGS. 3 through 6 show the structural advantage of animal hair as the additional material.

FIG. 3B is a photograph of a microscopic magnification of a rabbit hair 301. FIG. 3A is a schematic drawing of the photograph of FIG. 3B. As shown in these figures, the surface 302 comprises barbs 303 that contribute to the friction between the additional material—in the case of FIG. 3 a rabbit hair 301, and the chosen NOAH.

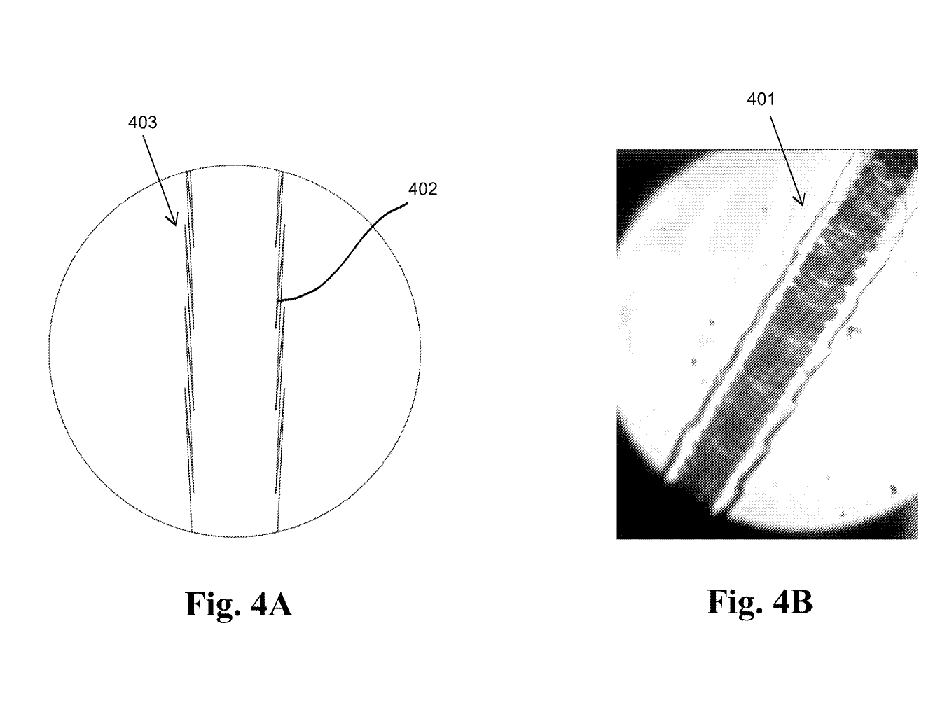

FIG. 4B is a photograph of a microscopic magnification of a cat hair 401. FIG. 4A is a schematic drawing of the photograph of FIG. 4B. The cat hair 401 also has barbs 403 on its surface 402, similar to the ones on the surface 302 of the rabbit hair 301, but in the case of the cat hair, they are longer.

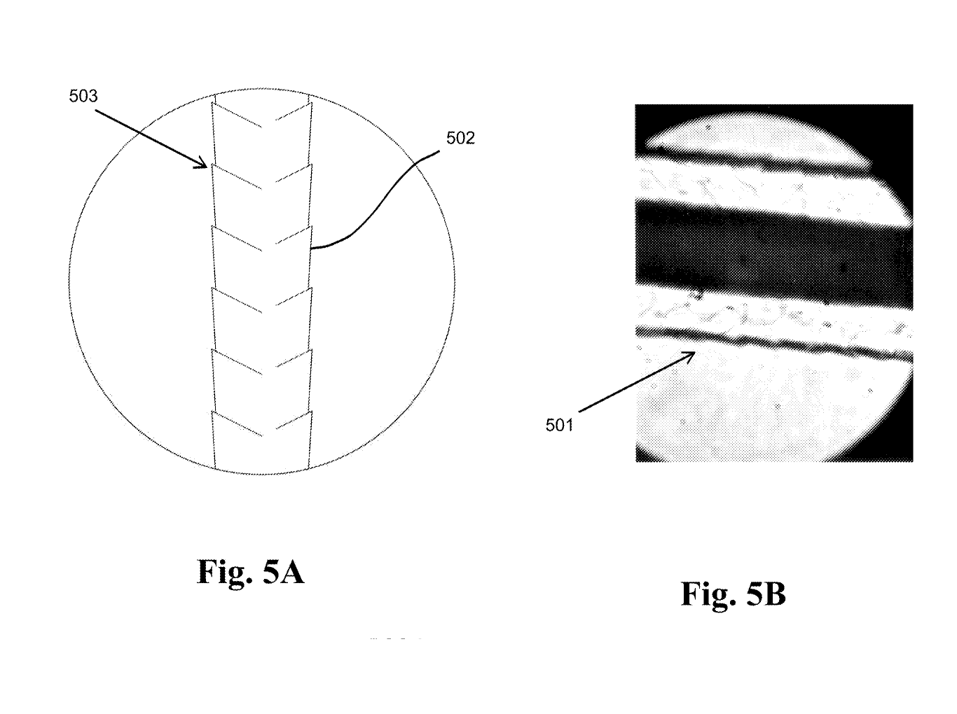

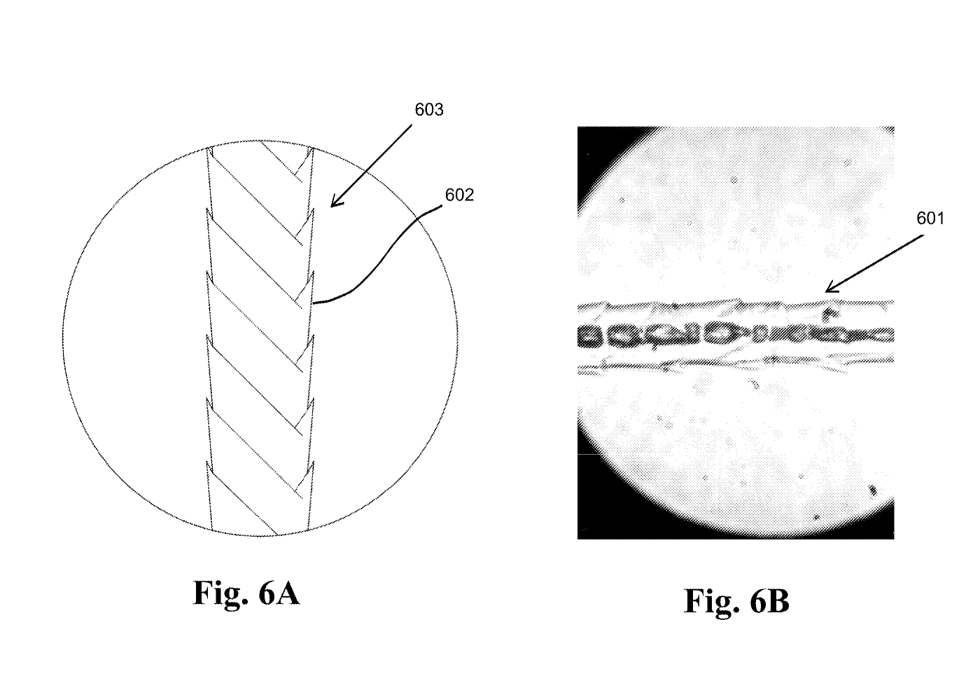

FIGS. 5B and 6B are both photographs of a microscopic magnification of dog hair, but from different types of dogs. FIGS. 5A and 6A are schematic drawings of the photographs of FIGS. 5B and 6B, respectively. FIG. 5 shows wider barbs 503 than the ones of FIGS. 3 and 4, and FIG. 6 shows deeper barbs 603 with greater gradient.

In the experiments performed by the inventors each of the animal hairs provided a satisfactory result, and different types of hair can affect the final appearance and can be chosen by the user, according to personal preferences, like in any other case where there is more than one option to a cosmetic product. Moreover, the additional material may comprise a combination of hair from different animals.

Although the description refers to cat, dog, and rabbit hair, it is not meant to limit the invention only to those types of hair, but these sources are only provided to illustrate embodiments of the invention. The hair can belong to any animal that has a suitable geometrical shape of the surface of the hair and, as said, can be replaced by synthetic filaments that mimic the surface-roughness of animal hair.

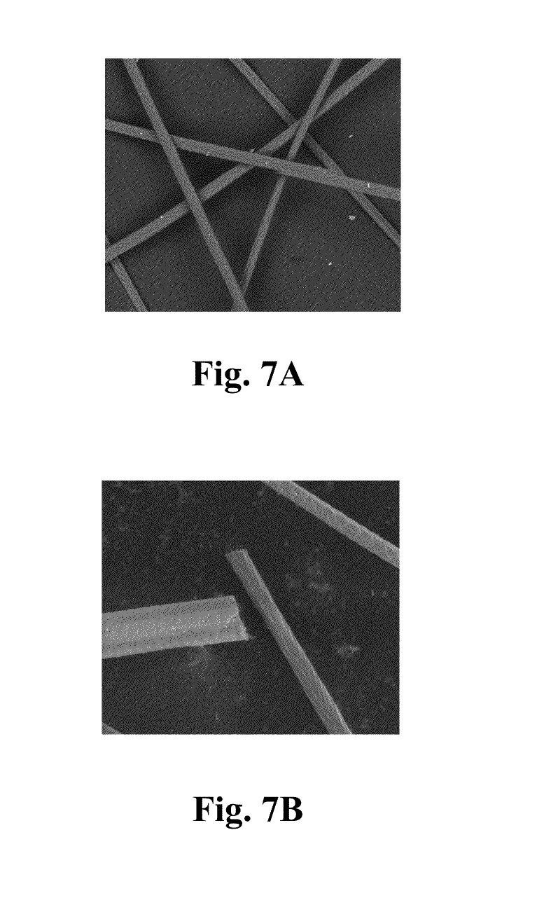

As can be seen from FIGS. 7A and 7B, which demonstrate features of rabbit hair, the hair is covered with scales (barbs), which are more elongated and present at an angle of approximately 30 degrees in thin hair (FIG. 7A), and more rounded in thick hair (FIG. 7B).

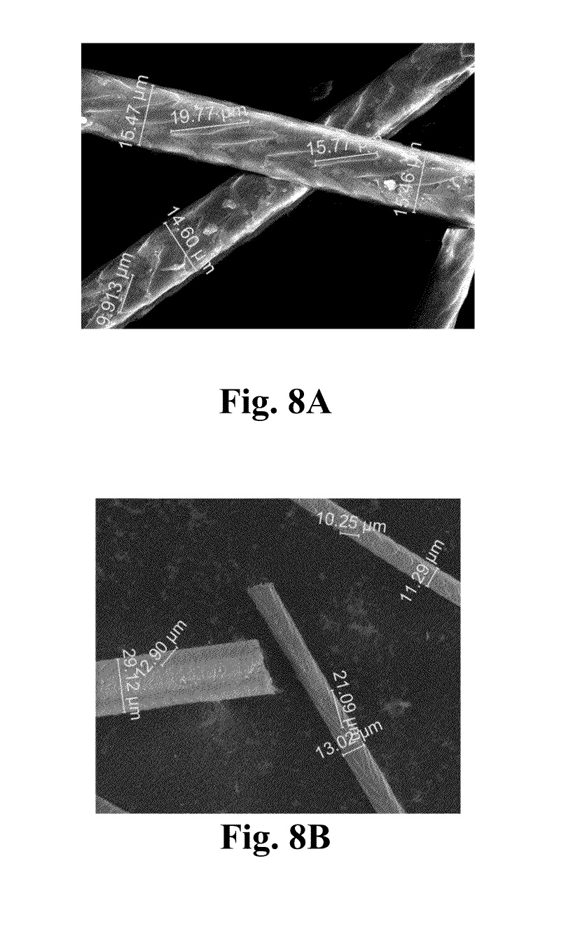

As can be seen from FIGS. 8A and 8B, the width of the thin hair filaments is between 14-16 μm, and the length of the scales (barbs) is between 9 and 20 μm (FIG. 8A). The width of the thick hair is between 11-30 μm, and the length of the scales (barbs) is between 10-22 μm (FIG. 8B). As can also be seen from FIGS. 7 and 8, the barbs are present at all sides of the hair filament, i.e., at all directions of, or all around, the hair filament, similar to the configuration in animal hair.

The synthetic fibers used according to the invention may have a length of about 1 to about 3 millimeters, although it should be understood that the length of those fibers does not have to be uniform.

In some embodiments, the thickness of the synthetic filaments that mimic the surface roughness of animal hair may be between about 10 to about 50 μm.

In other embodiments, the synthetic filaments have barbs which are between about 5-30, e.g., 5-10, 10-15, 15-20, 20-25, or 25-30, μm long. In particular such embodiments, the barbs are present at all sides of the synthetic filament surface and/or protruding from the synthetic filament surface before use.

Synthetic filaments as described above, which have specific length, thickness, and desired barbs pattern, and are thus capable of mimicking animal hair, can be prepared by standard methods in the field, including the methods described above for surface modifications of synthetic fibers.

The term “about” as used herein means that values that are 10% higher or 10% lower of the particular value referred to are also included.

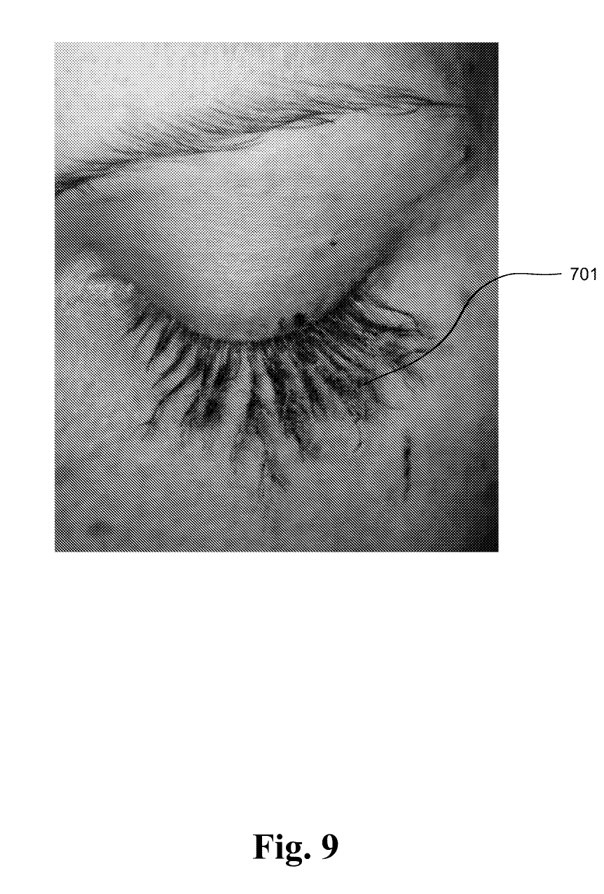

It was surprisingly found by experiments, that when first coloring the additional material the outcome improves significantly. FIG. 9 shows animal hair as the additional material 701 which was not colored. The color of the additional material 701 is obviously different than the eyelashes on which the additional material 701 is applied. In addition, the shape of the final structure is scattered unevenly.

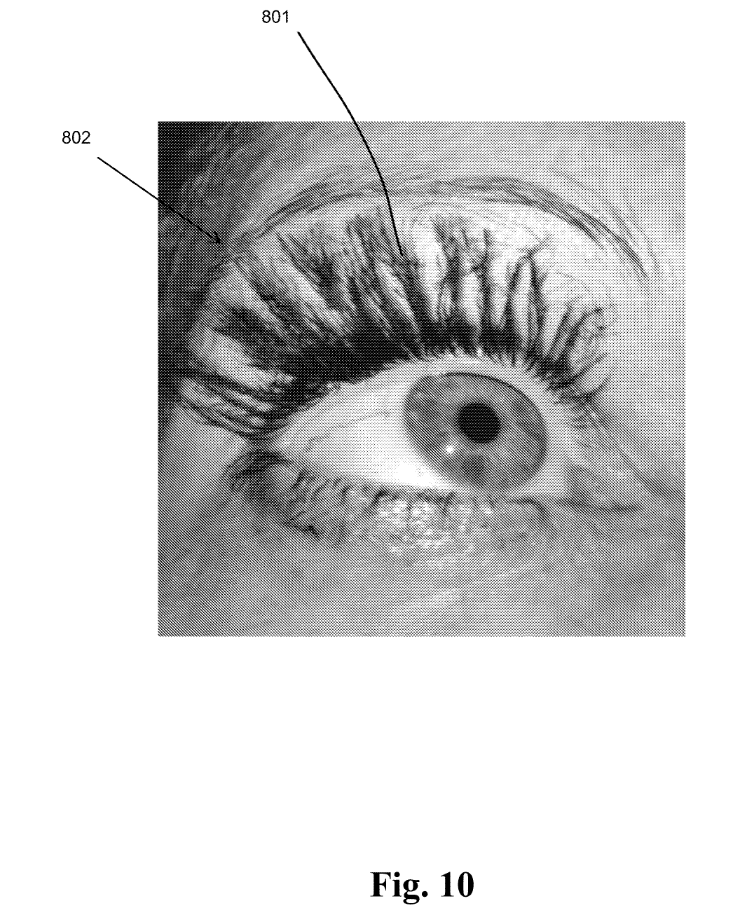

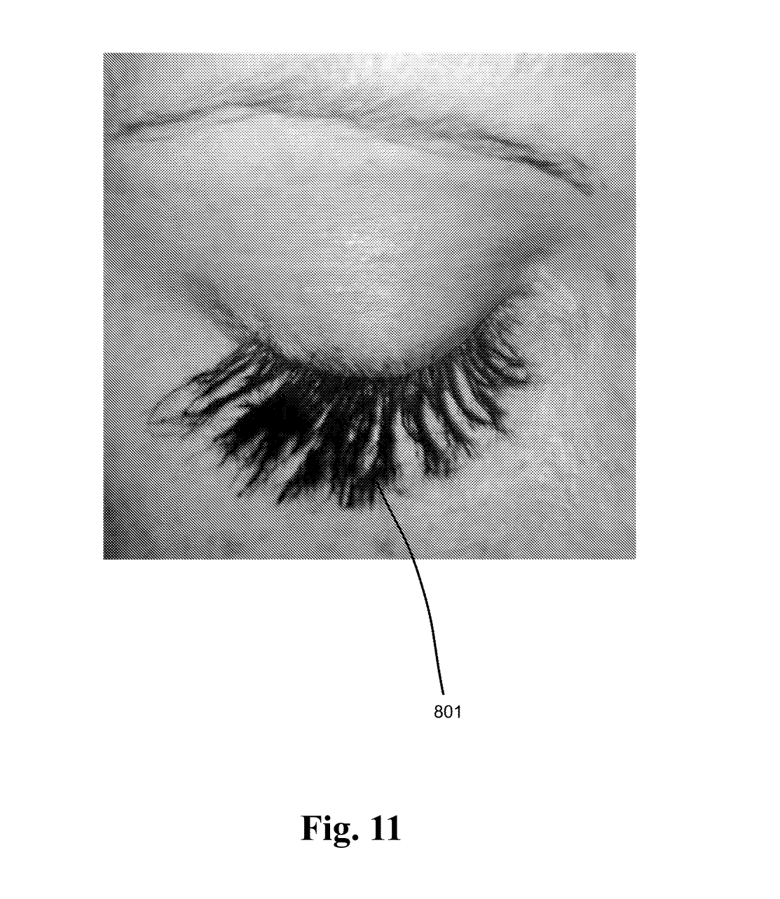

FIGS. 10 and 11 show a significant improvement in the result, when the additional material 801 is initially colored. Of course the chosen color does not have to be black, and can be chosen by the user to fit the color of the original NOAH. The colored additional material 801 is less fibrous than the additional material 701 of FIG. 9, and integrates with the NOAH to look as an extension of the NOAH.

Another advantage of the present invention is the fact that the length of the addition to the NOAH is nearly unlimited. The connection between the NOAH and the additional material, and between two layers of additional material, is so strong that it provides substantial endurance that permits the use of great amount of additional material. In FIG. 10 the final outcome 801 of the eyelashes shows they were extended by more than three times their original size. During experiments, measured length was at least 2.5 cm. As supported by the above experiments, when the animal hair is colored before use, it strengthens the connection between the animal hair and the NOAH. This finding has a significant influence on the durability of the product, and provides a substantial improvement over the prior art.

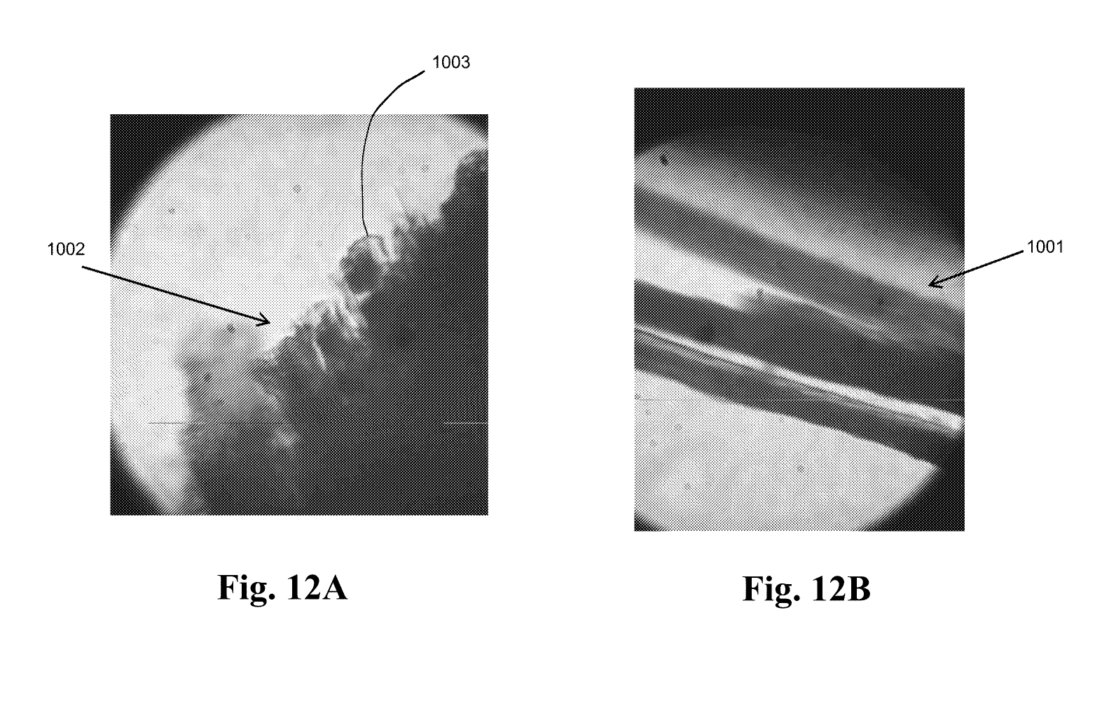

FIG. 12B is a photograph of a microscopic magnification of colored animal hair 1001 according to the examples, and FIG. 12A is another photograph of a microscopic magnification of a mixture of colored animal hair, showing the edges 1002 of the hair, wherein the surface clearly comprises barbs 1003 that provides the desired geometrical result.

After the completion of the process, the formed structure can be straightened, rounded, and shaped, even manually. The removal of the additional material from the NOAH is dependent on the adhesive material. When the adhesive material is for example, mascara, which is not water-proof, the removal can be performed be simply washing the NOAH. If for example, the mascara is water-proof, the removal can be performed with any conventional makeup removal substance.

It is noted that all the embodiments described in this application relate to any aspect of the invention to which they are applicable.

All the above description has been provided for the purpose of illustration and is not meant to limit the invention in any way. The invention presents significant advantages over the existing art. For example, the fact that the connection formed between the NOAH and the additional material is so strong, that the result can stay the same for at least two weeks, even when the extension is much greater than what can be achieved according to the prior art.

1. A system for extending and/or thickening natural or artificial hair (NOAH), comprising:
a) an adhesive material; and
b) an additional material suitable to be connected to said NOAH, which comprises animal hair and/or structurally-modified synthetic fibers the surface of which has been provided with roughening structures mimicking the surface of animal hair;
wherein said additional material is contained in a container.

2. The system of claim 1, wherein the NOAH are human hair.

3. The system of claim 2, wherein the human hair is eyelashes or eyebrows.

4. The system of claim 1, wherein the animal hair is selected from the group consisting of cat, dog, and rabbit hair.

5. The system of claim 1, wherein the animal hair is colored.

6. The system of claim 1, wherein said structurally-modified synthetic fibers have a thickness in a range of about 10 to about 50 μm.

7. The system of claim 1, wherein the surface of said structurally-modified synthetic fibers has roughening structures at all sides.

8. The system of claim 1, wherein said roughening structures have the shape of barbs protruding from said surface of said synthetic fibers.

9. The system of claim 1, wherein the adhesive material is mascara.

10. The system of claim 1, wherein the adhesive material and the additional material are mixed together.

11. The system of claim 1, further comprising a container for the adhesive material.

12. The system of claim 1, further comprising an applicator for the adhesive material and/or the additional material.

13. In a method for extending and/or thickening natural or artificial hair (NOAH), the improvement comprising using, as a material suitable to be connected to said NOAH, animal hair and/or structurally-modified synthetic fibers the surface of which has been provided with roughening structures mimicking the surface of animal hair.

14. A method for extending and/or thickening natural or artificial hair (NOAH), comprising applying to a NOAH an adhesive material and an additional material comprising animal hair and/or structurally-modified synthetic fibers the surface of which has been provided with roughening structures that mimic the surface of animal hair.

15. The method of claim 14, wherein said adhesive material and said additional material are mixed together prior to applying, and applied together as a mixture.

16. The method of claim 14, wherein the adhesive material is mascara.

17. The method of claim 14, wherein the additional material is colored before use.
